(19) United States
(12) Patent Application Publication
(10) Pub. No.: US 2007/0072940 A1
(43) Pub. Date: Mar. 29, 2007

(54) Use of quinol derivatives thereof for cosmetic, dietary supplement or pharmaceutical compositions and compositions thereby obtained.

(76) Inventor: Sal Abraham, Easton, PA (US)

Correspondence Address: SAL ABRAHAM, 1470 MOHAWK LANE, EASTON, PA, 18040, US

(21) Appl. No.: 11/162,943

(22) Filed: Sep. 28, 2005

(51) Int. Cl. A61K 31/355

(52) U.S. Cl. 514/458

(57) ABSTRACT

The present invention relates to a cosmetic, dietary supplement, or pharmaceutical composition for oral ingestion or topical application to the skin comprising effective amounts of a tocopherol quinone, tocopherol quinol, plastoquinol, menaquinol, or phylloquinol compound, or any combination thereof as a method for preventing free radical oxidation or preventing or treating environmental skin damage including photoaging, trauma, discoloration, dryness, thickness, elasticity, or wrinkles.

### BACKGROUND OF THE INVENTION

The present invention relates to the use of tocopherol quinone, tocopherol quinol, plastoquinol, menaquinol, or phylloquinol as a cosmetic, dietary supplement, or pharmaceutical compositions for oral ingestion or topical application to the skin, which is useful in preventing or treating the signs of environmental damage, aging in the skin, or free radical oxidation.

The skin is one of our most vital organs due to its role as barrier against a host of harmful and damaging environmental substances such as UVA/UVB light and various atmospheric pollutants. The first layer of the skin, which is the epidermis, absorbs the brunt of this daily borage of externally degrading components.

Ultraviolet (UV) light is probably the most common and well known environmental component that contributes to damage and premature aging in the skin. UV light or radiation primarily consists of UVA and UVB wavelengths, which possess similar and different actions when exposed to the skin. UVA is thought to be the less harmful UV wavelength but it has become known that UVA light promotes the formation of oxidants or free radicals in the skin which can cause damage to the elastin and collagen of connective tissue and thus leads to premature aging (i.e. photo aging) of the skin. UVB light is more harmful due to its role in the formation of oxidants or free radicals in the skin coupled with its ability to induce an acute inflammatory response (i.e. sunburn). Several of the effects of UVB light are thought to contribute skin carcinogenesis.

U.S. Pat. No. 5,378,461 by Neigut describes a composition for the topical treatment of skin damage. Neigut discloses that the invention provides a mixture of an oil-based carrier, such as squalane, a coenzyme Q.sub.4 to Q.sub.15, and vitamins A and E. Neigut states that the mixture forms a balm of any desired constituency, which can be applied directly to the site of skin damage, with extremely low risk of off-site toxicity and very little waste. Neigut describes that this invention has proven to be remarkably effective in penetrating the skin and treating many forms of skin damage, such as hypertrophic and keloid scar tissue, acute phototoxicity, hyperpigmented lesions, skin discoloration, keratosis, and skin hardening. This invention represents an improvement in standard cosmetic preparations for topical application to the skin due to the combination of ingredients. However, this combination is only intended to treat and not prevent the previously mentioned forms of skin damage, which is a direct limitation based upon its overall effectiveness. It is also limited to this specific combination and does not incorporate any superior quinol forms of antioxidants.

U.S. Pat. No. 6,068,848 by Gubernick et al. describes an antioxidant mixture compromising tocoherol. Gubernick describes a composition comprising of at least one of each of the antioxidants selected from the group consisting of (a) tocopherol and derivatives thereof, (b) ascorbic acid and derivatives thereof, (c) a butylated phenol, (d) N-acetyl cysteine, (e) a rosemary extract, and (f) ubiquinone or a derivative thereof. Gubernick discloses that the combination of antioxidants of the present compositions have now been unexpectedly shown to be capable of preventing the development or worsening of skin damage resulting from actual chronic sun exposure. This invention represents an improvement in standard cosmetic preparations for topical application to the skin due to the combination of ingredients. However, this combination is limited to the previous mentioned ingredients and does not incorporate any superior quinol forms of antioxidants.

U.S. Pat. No. 6,521,668 by Anderson et al. describes a cosmetic composition and method of use. Anderson et al. discloses a composition comprising an essential antioxidant hesperetin, tetrahydrocurcumin, tetrahydrodemethoxycurcumin, or tetrahydrobisdemethoxycurcumin, or mixtures thereof. Anderson et al. describes these compositions have a number of benefits in connection with care of and prevention of damage to keratinous tissue. It is a further object of the invention to provide such compositions that are a strong defense against environmental aggressors such as smoke, smog, ozone, atmospheric pollutants, free radicals and ultraviolet radiation. This invention represents an improvement in standard cosmetic preparations for topical application to the skin. However, this invention is limited to the previously mentioned ingredients and does not incorporate any superior quinol forms of antioxidants.

U.S. Pat. No. 6,740,338 by Chopra describes a reduced form of coenzyme Q in high bioavailability stable dosage forms and related applications. This invention relates to novel storage stable compositions in dietary supplement, cosmetic or pharmaceutical dosage form (preferably oral dosage form) comprising effective amounts of ubiquinol, a reduced form of coenzyme Q, in combination with an amount of a lipid soluble reducing agent effective to maintain ubiquinol in its reduced state when preferably formulated in a soft gelatin capsule. These compositions can be used to treat numerous conditions or disease states including, for example, those conditions or disease states which are associated with oxidative tissue damage or mitochondrial dysfunction, including reduced mitochondrial oxidative function, mitochondrial encephalomyolopathies, cardiomyopathies, heart disease, especially including congestive heart failure, ischemia/reperfusion tissue damage, neurodegenerative disorders, including Alzheimer's disease, dementia and Parkinson's disease, high blood pressure, periodontal disease, a weakened immune system and high cholesterol or high triglycerides, among numerous others. This invention represents an improvement in standard pharmaceutical preparations for oral applications. This invention also represents an improvement in standard cosmetic preparations for topical application to the skin due to the use of ubiquinol, a reduced form of coenzyme Q. However, this invention neglects to mention or include the use of other various reduced forms of quinones such as tocopherol quinol, plastoquinol, menaquinol, or phylloquinol which all possess superior antioxidant properties for oral or topical administration.

### SUMMARY OF INVENTION

The present invention relates to cosmetic, dietary supplement, or pharmaceutical compositions for oral ingestion or topical application to the skin in order to prevent or treat environmental damage, aging in the skin, or free radical oxidation. There has been ongoing research demonstrating that the oral ingestion and topical application of various antioxidants prevents the environmental damages that cause the skin to wrinkle, lose elasticity, dry and discolor. The primary action is thought to be the quenching of free radicals and a decrease in the lipid peroxidation in the skin that is accelerated by UV light and various atmospheric pollutants.

The object of the present invention is to provide stable compositions of new and useful forms of antioxidant quinols. The introduction of new and improved antioxidants into the market place represents useful applications to improve the general health and appearance of the population. The present invention is novel, unobvious, and superior to the prior art and all previous mentioned inventions. Stable compositions for commercial use of the reduced forms of alpha tocopherol, platoquinone, menaquinone, or phylloquinone for oral ingestion or topical application individually or in combination have never been previously disclosed or sold as commercial preparations. These reduced forms or quinol analogs possess superior antioxidant properties and therefore result in superior prevention and treatment of environmental damage, aging in the skin, and provide health benefits associated with the quenching of free radicals.

### DETAILED DESCRIPTION

The general chemical heading of vitamin quinol analog represents the reduced form of various quinones. Quinols are diacid phenols while quinones are aromatic dicarbonyl compounds derived from dihydroxy aromatic compounds. The biosynthetic pathway of the compounds of this invention result in the formation of quinone end products. For example the biosynthesis of Vitamin E (Alpha tocopherol) and Vitamin K3 (Menadione) results in the formation of alpha-tocopherol quinone and menaquinone. Until most recently the quinone forms of vitamin K and CoQ10 have been the predominate forms utilized in various cosmetic and pharmaceutical preparations. Vitamin E has been predominately in the D-alpha tocopherol acetate form and now more recently in the D-alpha tocopherol phosphate form. To the best of my knowledge, I propose for the first time a composition for the use of the reduced form of alpha tocopherol quinone, platoquinone, menaquinone, or phylloquinone for oral ingestion or topical application for the prevention and treatment of environmental skin damage or free radical oxidation.

The quenching of free radicals and a decrease in the lipid peroxidation in the skin is thought to be the primary mechanism in which past and present compounds exert the prevention and treatment of environmental skin damage. Leibler DC, et al. Carcinogenesis 2000 Feb; 21(2):221-5 demonstrates that epidermal Vitamin E (alpha tocopherol) status plays vital role in UVB induced skin damage and carcinogenesis in mice. Mice were either exposed to a single dose or repeated doses of UVB light at doses typical of inducing photocarcinogenesis. Epidermal alpha tocopherol levels increased gradually with the repeated UVB light exposure. Topical application of proxidant chemical resulted in the accumulation of alpha tocopherol and its oxidation products alpha tocopherol quinone and alpha tocopherol quinol. The researchers concluded that UVB and tumor promoting chemicals all exert qualitatively different effects on epidermal alpha tocopherol status.

Vitamin E or Alpha tocopherol (Merck Index, 12th edition, reference 10159, page 1712) is a fat soluble vitamin that functions solely as a membrane bound antioxidant that prevents cell membrane damage by inhibiting peroxidation of membrane phospholipids and disrupting free radical chain reactions induced by formation of lipid peroxides. Alpha tocopherol is essentially used for controlling vitamin E deficiencies or as a nutritional factor, especially for controlling muscle degeneration.

Alpha tocopherol esters have been described, in particular the succinate, the nicotinate or the acetate. The synthesis of alpha tocopherol acetate is also described in U.S. Pat. No. 2,723,278 and that of other esters are described in the document J. Amer. Chem. Soc. (1943) 65, 918-924.

DL alpha tocopherol phosphate is also known (see P. KARRER et al., Helv. Chim. Acta (1940) 23, 1137-8), as is its action on muscle metabolism (see J. Biol. Chem. 1942, 146, pages 309-321). Another document describes the biological role as an antioxidant on brain tissue (Biol. Antioxidants Trans., 1st Conf., 1946, pages 61-62). An anticoagulant action through an action on the polymerization of fibrin has also been described (Can. J. Biochem. and Physiol. 1959, 37, pages 501-505). An antimicrobial action in vitro on B. subtilis and S. aureus has also been described (Naturwissenschaften 1960, 47, page 17).

U.S. Pat. No. 5,387,579 by Meybeck et al. describes a use of alpha tocopherol phosphate or a derivative thereof for preparing cosmetic, dermatological or pharmaceutical compositions, and compositions thereby obtained for the prevention or treatment of allergic manifestations such as skin allergy or bronchial asthma, or inflammatory manifestations, or for the prevention or treatment of the harmful effects of free radicals.

Alpha tocopherol quinone and quinol are known as described by Mackenzie et al. J Biol Chem 1949; 183(2)655-63. Mackenzie et al. demonstrated that injectable and oral tocopherol quinol could prevent and treat nutritional muscular dystrophy in the rabbit. They also found that tocopherol quinol could prevent creatinuria and loss of weight caused by vitamin E deficiency.

Endogenous production of alpha tocopherol quinone and quinol is known as described by Hughes and Tove J Biol Chem 1980; 255(10): 4447 which from a regulatory standpoint allows for the commercial sale as a dietary supplement. Kohar I, et al. Free Radic Biol Med 1995 Aug; 19(2):197-207 demonstrates that alpha tocopherol can be oxidized in many biological systems to its corresponding quinone and quinol analogs. Bindoli A, et al. Biochem Int 1985 May; 10(5):753-61 demonstrates the antioxidant ability of alpha tocopherol quinone and alpha tocopherol quinol. Both compounds were shown to inhibit lipid peroxidation while alpha tocopherol quinol exhibited superior inhibition of lipid peroxidation in liposomes.

Neuzil et al. Proc Natl Acad Sci 1997 July; 94:7885-90 demonstrates the ability of alpha tocopherol quinol in vitro as an efficient multifunctional inhibitor of radical initiated oxidation of low density lipoprotein lipids. They also discuss that their results combined with previous research suggest that alpha tocopherol quinone and alpha tocopherol quinol are non-toxic and that humans can reduce the quinone to the quinol. The three main results of the research support the superior antioxidant properties of alpha tocopherol quinol. The first result demonstrates that alpha tocopherol quinol has the ability to readily associate with LDL and instantaneously reduce the lipoprotein ubiquinone to ubiquinol thereby maintaining antioxidant activity. Second, alpha tocopherol quinol directly intercepted aqueous peroxyl radicals. Third, alpha tocopherol quinol rapidly quenched alpha tocopheroxyl radicals in oxidizing LDL. Similar antioxidant activities were seen with alpha tocopherol quinol when added to HDL or protein free intralipid indicating no dependence upon lipoprotein or protein.

Plastoquinol is the reduced form of plastoquinone-9 and a polyunsaturated side-chain quinone derivative, which is an important link in the electron transport chain of green plants during the photosynthetic conversion of light energy by photophosphorylation into the potential energy of chemical bonds. There are several plastoquinones with side chains of different length in position 5. They are designated as plastoquinone-n where n is the number of carbon atoms in the side chain where n indicates the number of isoprenoid units. Kruk J, et al. Free Radic Res 1994 Nov-Dec; 21(6):409-16 demonstrates the free radical scavenging ability of plastoquinol and several other prenylquinones. Plastoquinol and alpha tocopherol quinol were shown to possess a free radical scavenging ability greater than that of ubiquinol and alpha tocopherol.

Vitamin K (Phytonadione) is a fat soluble vitamin that was first identified as essential factor for blood clotting. Vitamin K functions in the synthesis of osteocalcin, a key protein required in bone formation. Vitamin K when applied topically appears to have the ability to reduce the severity of skin trauma such as bruising or blemishes as demonstrated by Shah NS, et al. J Am Acad Dermatol 2002 Aug; 47(2):241-4 and Lou WW, et al. Dermatol Surg 1999 Dec 25:12; 942-4. Vitamin K1 (Phylloquinone) and Vitamin K2 (Menaquinone) both possess antioxidant properties. Kruk J, et al. Free Radic Res 1994 Nov-Dec; 21(6): 409-16 discusses the antioxidant properties of the reduced forms vitamin K1 (Phylloquinol) and K2 (Menaquinol). Phylloquinol and Menaquinol were both shown to possess a free radical scavenging ability greater than that of alpha tocopherol quinol, plastoquinol, and alpha tocopherol.

For oral application, the mixture can be combined with pharmaceutically acceptable carriers such as magnesium stearate, cellulose derivatives, and a reducing agent, which is selected from the group consisting of reduced glutathione, L-cysteine, N-acetyl cysteine, reduced alpha lipoic acid, tocotrienols, tocopherols, vitamin E, vitamin E esters, vitamin c, vitamin c esters, vitamin A (retinol, retinoic acid), vitamin A esters, alpha carotene, beta carotene, lutein, zeaxanthin, astaxanthin, lycopene, flavonoids, L-carnitine, acetyl L-carnitine, propionyl L-carnitine, magnesium, zinc, selenium, manganese, riboflavin, niacinamide, curcuminoids, proanthocyanidins, NADH, NADPH, resveratrol, bilberry extract, milk thistle extract, retinol, retinoic acid, retinoic acid esters, retinal, omega-3-fatty acids and mixtures, thereof. The carrier may be in any form appropriate to the mode of delivery, for example hard and soft gelatin capsules, aquicaps, tablets, caplets, and liquids in the form of sprays and pumps via intranasal or intra oral.

For topical application, the antioxidant mixture can be combined with cosmetically and/or pharmaceutically acceptable carriers. The term “pharmaceutically or cosmetically acceptable carrier” refers to a vehicle, for either pharmaceutical or cosmetic use, which vehicle delivers the active components to the intended target and which will not cause harm to humans or other recipient organisms. As used herein, “pharmaceutical” or “cosmetic” will be understood to encompass both human and animal pharmaceuticals or cosmetics. Useful carriers include, for example, water, acetone, ethanol, ethylene glycol, propylene glycol, butane-1,3-diol, isopropyl myristate, isopropyl palmitate, or mineral oil. Methodology and components for formulation of cosmetic and pharmaceutical compositions are well known, and can be found, for example, in Remington's Pharmaceutical Sciences, Eighteenth Edition, A. R. Gennaro, Ed., Mack Publishing Co. Easton Pa. 1 990. The carrier may be in any form appropriate to the mode of delivery, for example, solutions, colloidal dispersions, emulsions(oil-in-water or water-in-oil), suspensions, creams, lotions, gels, foams, mousses, sprays and the like.

The formulation, in addition to the carrier and the antioxidant mixture, also can comprise other components, which may be chosen depending on the carrier and/or the intended use of the formulation. Additional components include, but are not limited to, water soluble colorants (such as FD&C Blue #1); oil soluble colorants (such as D&C Green #6); water soluble sunscreens (such as Eusolex 232); oil soluble sunscreens (such as Octyl Methoxycinnamate); particulate sunscreens (such as Zinc Oxide); organic sunscreens (such as para-amino benzoic acid and its esters, benzophenones, phenyl or homomenthyl salicylates, and cinnamates); antioxidants (such as BHT); chelating agents (such as Disodium EDTA); emulsion stabilizers (such as carbomer); preservatives (such as Methyl Paraben); fragrances (such as pinene); flavoring agents (such as sorbitol); humectants (such as glycerine); waterproofing agents (such as PVP/Eicosene Copolymer); water soluble film-formers (such as Hydroxypropyl methylcellulose); oil-soluble film formers (such as Hydrogenated C-9 Resin); cationic polymers (such as Polyquaternium 10); anionic polymers (such as xanthan gum); vitamins (such as Tocopherol); and the like. As will be apparent, the composition can be a therapeutic product the antioxidants being the sole actives, or in combination with other actives. However, the composition can also be a makeup product, for example, a lipstick, foundation, concealer, bronzer, blush, eye shadow and the like.

The reduced forms of alpha tocopherol quinone, platoquinone, menaquinone, or phylloquinone possess superior antioxidant properties as demonstrated by the previous research. Thus the quinol analogs can be administered daily by oral ingestion or topical application individually or in combination to the skin for reducing lipid peroxidation in the skin from UV light or environmental pollutants. Therefore this composition represents an improvement in standard cosmetic, dietary supplement, or pharmaceutical compositions.

After an extensive review of the scientific literature regarding these novel and unobvious quinol analog compositions and their superior antioxidant function, it then became the focus of this invention that the quinol analogs could be administrated perorally or topically for preventing and treating the signs of environmental damage or aging in the skin in mammals by reducing lipid peroxidation in the skin from UV light or environmental pollutants. The oral daily doses can be between 1 mcg to 9000 mg./day. The preferred daily dosing schedule should be divided into 3 sub dose applications per day. It is suggested as an example that topical application of the composition, in an amount of from about 0.1 .mu.g/cm.sup.2 to 2 mg/cm.sup.2 of exposed skin, be performed from about once per week to about 4 or 5 times daily, preferably from about 3 times a week to about 3 times daily, most preferably about once or twice per day. By “chronic” application, it is meant herein that the period of topical application may be over the lifetime of the user, preferably for a period of at least about one month to about twenty years, thereby resulting in the treatment or prevention of the external signs of photo aging.

The foregoing descriptions of the invention are for illustration only. Modifications not included in the description, which are obvious to those skilled in the art, are intended to be included in the scope of the following claims.

### EXAMPLE 1

Alpha tocopherol quinol Intensive Anti-Wrinkle Soft Cream
Active Ingredient: D-Alpha tocopherol quinol
Base Ingredients: Sorbitan Monooleate NF
Glycerin USP
Polysorbate 80 NF
Ascorbyl palmitate (% ascorbyl palmitate>% of Alpha tocopherol quinol in sample)
Medium Chain Triglycerides
IMWITOR 370 (Glyceryl Stearate Citrate)
MIGLYOL 812 (Caprylic/Capric Triglyceride)
SOFTISAN 601 (Glycerol Cocoate and Hydrogenated Cocoanut Oil and Ceteareth-25)
EWALAN ODE-50 (Octyldodecyl Lanolaet)
Pronalen Fuir Acid AHA-5 (Lemon and Passion Fruit Concentrate)
Preservative
Purified Water
Perfume Exemplary

Procedure: mix sorbitan monooleate NF, polysorbate 80 NF, glycerin and medium chain triglycerides in a suitable jacketed mixing vessel. After mixing the above components, the mixture is raised in temperature to about 55 C ( 5 ) while mixing constantly. 0.1% to about 10% of D-alpha tocopherol quinol is then added to the above heated mix while stirring. The alpha tocopherol quinol is thoroughly mixed into solution at elevated temperature for a period of from 1-2 hours. The remaining components are added thereafter at elevated temperature in order to ensure a uniform product. The product (a cream) may be packaged using standard packaging procedures.

### EXAMPLE 2

Oral Dosage Form of Alpha tocopherol quinol w/Surfactants
   This example sets forth a composition and method for providing a soft gelatin capsule (soft gel) of the active ingredient D-Alpha tocopherol quinol.
   Procedure:
Mix the following components in a suitable jacketed mixing vessel:
Span 80 (1-15%);
Glycerine, propylene glycol or other suitable polyhydric alcohol (1%-15%);
Tween80 (20% to 90%);
Medium Chain Triglycerides (MCT, 5% to 25%).
After mixing the above components, the mixture is raised in temperature to about 55.degree. C. (.plus or minus.5.degree.) while mixing constantly 0.5% to about 10% of D-Alpha tocopherol quinol is then added to the above heated mix while stirring. The D-Alpha tocopherol quinol is thoroughly mixed into solution at elevated temperature for a period of from 1-2 hours. Then ascorbyl palmitate is added in an effective amount ranging from about 1% to about 15% by weight and the mixture at elevated temperature is stirred for 1 to 2 hours. The mixing vessel is then connected to a cooling system (cooled water) and while mixing, the heated mixture is cooled to room temperature (about 23.degree. C. .plus or minus.3.degree.). After the liquid is cooled to room temperature, the mixer is shut down and the cooling water is disconnected. The liquid is then transferred to a suitable stainless steel drum and the empty space in the drum is flushed with nitrogen. The drum is then sealed. The finished liquid is analyzed using HPLC using an electrochemical detector for quantitative determination of alpha tocopherol quinone and alpha tocopherol quinol. The liquid is thereafter encapsulated in soft gelatin capsules containing an opacifier (TiO.sub.2) and colorant utilizing standard manufacturing procedures.

The final soft gelatin capsule has the following components (in percent by weight excluding gelatin capsule):

| | |
|---|---|
| Span 80 | 5% |
| Glycerine | 4% |
| Tween 80 | 65% |
| MCT | 18% |
| D-Alpha Tocopherol quinol | 4% |
| Ascorbyl Palmitate | 4% |

### EXAMPLE 3

Oral Dosage Form of D-alpha Tocopherol Quinol for Hard Gelatin Capsule

100 IU of D-alpha tocopherol quinol
   Excipients include 10 mg dicalcium phosphate and 5 mg magnesium stearate The powder is then encapsulated utilizing standard manufacturing principals.

What is claimed is:

1. A method for treating or preventing the symptoms of environmental skin damage or free radical oxidation comprising administering a composition of matter comprising a cosmetic, dietary supplement, or pharmaceutical composition comprising as active ingredient an efficient amount of a tocopherol compound selected from the group consisting of:
a) tocopherol quinone, tocopherol quinol, or any isomer or analog thereof or any combination thereof,
b) a DL, D, or L form of said tocopherol compound,
c) an alpha, beta, gamma, or delta form of said tocopherol compound,
d) a cosmetically or pharmaceutically acceptable ester, salt, phosphoester, or ether of said tocopherol compound or any combination thereof.

2. The composition of claim 1, wherein said tocopherol compound is incorporated in a cosmetically or pharmaceutically acceptable excipient to constitute a composition, said tocopherol compound being in a weight concentration ranging from 0.001 to 90% with respect to the total weight of said composition.

3. The method of claim 1, wherein said symptoms of environmental skin damage or aging are selected from the group consisting of photo aging on the skin, lines and wrinkles on the skin, discoloration on the skin, trauma on the skin, loss of elasticity in the skin, skin thinning, or skin dryness.

4. The method of claim 1, wherein said administration is selected from the group consisting of oral, topical, transdermal, rectal, intranasal, sublingual, or parenteral.

5. A method for treating or preventing the symptoms of environmental skin damage or free radical oxidation comprising administering a composition of matter comprising a cosmetic, dietary supplement, or pharmaceutical composition comprising as active ingredient an efficient amount of a plastoquinone compound selected from the group consisting of:
a) plastoquinol or any isomer or analog thereof or any combination thereof,
b) a cosmetically or pharmaceutically acceptable ester, salt, phosphoester, or ether of said plastoquinol compound or any combination thereof, or in any combination with claim 1.

6. The composition of claim 5, wherein said plastoquinol compound is incorporated in a cosmetically or pharmaceutically acceptable excipient to constitute a composition, said plastoquinol compound being in a weight concentration ranging from 0.001 to 90% with respect to the total weight of said composition.

7. The method of claim 5, wherein said symptoms of environmental skin damage or aging are selected from the group consisting of photo aging on the skin, lines and wrinkles on the skin, discoloration on the skin, trauma on the skin, loss of elasticity in the skin, skin thinning, or skin dryness.

8. The method of claim 5, wherein said administration is selected from the group consisting of oral, topical, transdermal, rectal, intranasal, sublingual, or parenteral.

9. A method for treating or preventing the symptoms of environmental skin damage or free radical oxidation comprising administering a composition of matter comprising a cosmetic, dietary supplement, or pharmaceutical composition comprising as active ingredient an efficient amount of a menaquinone compound selected from the group consisting of:
a) menaquinol or any isomer or analog thereof or any combination thereof,
b) a cosmetically or pharmaceutically acceptable ester, salt, phosphoester, or ether of said menaquinol compound or any combination thereof or in any combination with claim 1 or 5.

10. The composition of claim 9, wherein said menaquinol compound is incorporated in a cosmetically or pharmaceutically acceptable excipient to constitute a composition, said menaquinol compound being in a weight concentration ranging from 0.001 to 90% with respect to the total weight of said composition.

11. The method of claim 9, wherein said symptoms of environmental skin damage or aging are selected from the group consisting of photo aging on the skin, lines and wrinkles on the skin, discoloration on the skin, trauma on the skin, loss of elasticity in the skin, skin thinning, or skin dryness.

12. The method of claim 9, wherein said administration is selected from the group consisting of oral, topical, transdermal, rectal, intranasal, sublingual, or parenteral.

13. A method for treating or preventing the symptoms of environmental skin damage or free radical oxidation comprising administering a composition of matter comprising a cosmetic, dietary supplement, or pharmaceutical composition comprising as active ingredient an efficient amount of a phylloquinone compound selected from the group consisting of:
a) phylloquinol or any isomer or analog thereof or any combination thereof,
b) a cosmetically or pharmaceutically acceptable ester, salt, phosphoester, or ether of said phylloquinol compound or any combination thereof or in any combination with claim 1, 5, or 9.

14. The composition of claim 13, wherein said phylloquinol compound is incorporated in a cosmetically or pharmaceutically acceptable excipient to constitute a composition, said phylloquinol compound being in a weight concentration ranging from 0.001 to 90% with respect to the total weight of said composition.

15. The method of claim 13, wherein said symptoms of environmental skin damage or aging are selected from the group consisting of photo aging on the skin, lines and wrinkles on the skin, discoloration on the skin, trauma on the skin, loss of elasticity in the skin, skin thinning, or skin dryness.

16. The method of claim 13, wherein said administration is selected from the group consisting of oral, topical, transdermal, rectal, intranasal, sublingual or parenteral.

17. A method of increasing tocopherol quinol in humans or animals by the administration of an effective amount of tocopherol quinone or any isomer or analog thereof or a salt, ester, phosphoester, or ether thereof or any combination thereof.

18. The method of claim 17, wherein said administration is selected from the group consisting of oral, topical, transdermal, rectal, intranasal, sublingual or parenteral.

19. A method of increasing plastoquinone in humans or animals by the administration of an effective amount of plastoquinol or any isomer or analog thereof or a salt, ester, phosphoester, or ether thereof or any combination thereof.

20. The method of claim 19, wherein said administration is selected from the group consisting of oral, topical, transdermal, rectal, intranasal, sublingual, or parenteral.

21. A method of increasing menaquinone in humans or animals by the administration of an effective amount of menaquinol or any isomer or analog thereof or a salt, ester, phosphoester, or ether thereof or any combination thereof.

22. The method of claim 21, wherein said administration is selected from the group consisting of oral, topical, transdermal, rectal, intranasal, sublingual or parenteral.

23. A method of increasing phylloquinone in humans or animals by the administration of an effective amount of phylloquinol or any isomer or analog thereof or a salt, ester, phosphoester, or ether thereof or any combination thereof.

24. The method of claim 23, wherein said administration is selected from the group consisting of oral, topical, transdermal, rectal, intranasal, sublingual, or parenteral.
